(19) United States
(12) Patent Application Publication
(10) Pub. No.: US 2014/0257142 A1
(43) Pub. Date: Sep. 11, 2014

(54) SENSOR FOR RELIABLE MEASUREMENT OF JOINT ANGLES

(71) Applicants: Thompson Sarkodie-Gyan, El Paso, TX (US); Huiying Yu, El Paso, TX (US); Noe Vargas Hernandez, El Paso, TX (US); Murad Alaqtash, El Paso, TX (US)

(72) Inventors: Thompson Sarkodie-Gyan, El Paso, TX (US); Huiying Yu, El Paso, TX (US); Noe Vargas Hernandez, El Paso, TX (US); Murad Alaqtash, El Paso, TX (US)

(21) Appl. No.: 13/790,780

(22) Filed: Mar. 8, 2013

(51) Int. Cl. A61B 5/11; A61B 5/00

(52) U.S. Cl. CPC A61B 5/1121; A61B 5/4528; A61B 5/1128; A61B 5/72; A61B 5/0013
USPC 600/595

(57) ABSTRACT

A sensor apparatus, system, and computer-usable non-transitory storage device storing program instructions for the measurement of the individual joint angles in dimensional space are disclosed. The joint angle measurements are measured in electrical quantities to represent the amplitude of joint movement in respect to a neutral position. Angular rate inertial sensors are attached to the body segments of interest. The output of the inertial sensors are fed into a precision differential amplifier and integrated. Two sequential filters are used for processing the output signals. Embodiments measure three-dimensional angles in real-time.

### CROSS REFERENCE TO RELATED APPLICATIONS

This patent application claims the benefit under 35 U.S.C. §119(e) of U.S. Provisional Patent Application Ser. No. 61/608,432 filed on Mar. 8, 2012 and entitled “SENSOR FOR RELIABLE MEASUREMENT OF JOINT ANGLES:' which is hereby incorporated by reference in its entirety.

### FIELD OF THE INVENTION

The disclosed embodiments relate to a goniometer sensor apparatus. The disclosed embodiments also relate to measuring individual joint angles in three-dimensions. The disclosed embodiments further relate to calculate the amplitude of joint movement respective to a neutral position.

### BACKGROUND

A growing marker has developed for tools and systems that track humans and other bodies at rest and in motion. The applications for such systems vary considerably, including such areas, for example, as the creation of computer-generated and graphical animations, the analysis of human-computer interaction, the assessment of performance athletics and other biomechanics activities, and the evaluation of workplace and other activities for general ergonomical fitness.

In general, range of motion is the angular movement of one body portion connected to or associated with a joint to that of a second body portion also associated with the same joint. Joint angle measuring devices, such as goniometers, measure a range of motion of joints in the body. Inaccurate proposed joint measuring solutions, however, limit the reliability of measurements taken from prior proposed goniometers. For example, the orientation on a table, chair or other object of a body part can be different between patients or can vary between the same person when taken at different points in time. The joint measuring device may be aligned improperly by a health care provider during a measurement. Human error in measuring joint angles is undesirable.

Therefore, a need exists for an improved sensor for reliable and accurate measurement of joint angles.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide an improved goniometer sensor apparatus.

It is another aspect of the disclosed embodiments to measure individual joint angles in three-dimensions.

It is a further aspect of the disclosed embodiments to calculate the amplitude of joint movement respective to a neutral position.

The above and other aspects can be achieved as is now described. A sensor apparatus, system, and computer-usable non-transitory storage device storing program instructions for the measurement of the individual joint angles in dimensional space are disclosed. The joint angle measurements are measured in electrical quantities to represent the amplitude of joint movement in respect to a neutral position. Angular rate inertial sensors are attached to the body segments of interest. The output of the inertial sensors are fed into a precision differential amplifier and integrated. Two sequential Butterworth filters are used for processing the output signals. Embodiments measure three-dimensional angles in real-time.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates a block diagram of a data-processing apparatus, which can be utilized for measuring joint angles, in accordance with a preferred embodiment;

FIG. 2 illustrates an exemplary graphical user interface for display of joint angle measurements, according to an embodiment; and

FIG. 3 illustrates an exemplary block diagram displaying integration of the difference between two angular rate gyroscopes to provide three-dimensional joint angles in real-time, according to an embodiment.

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

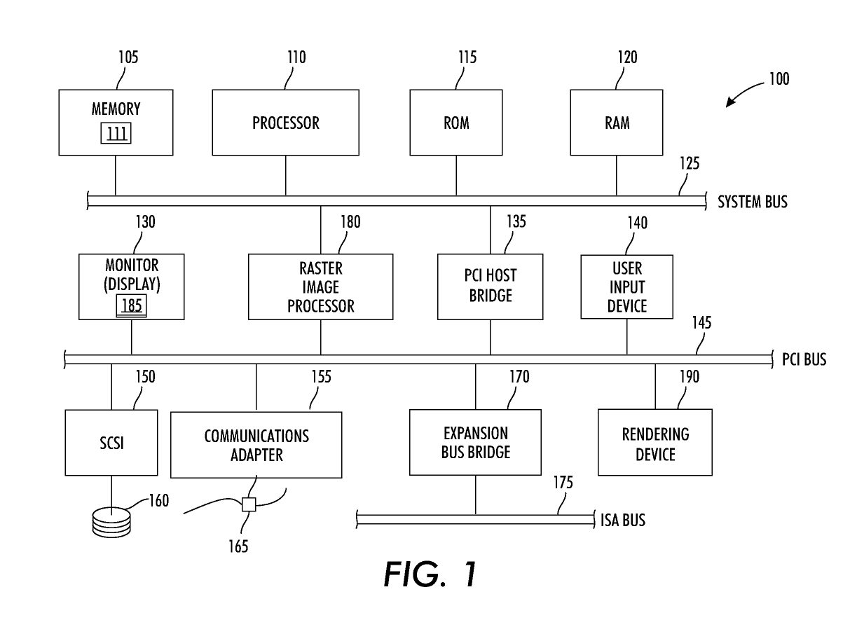

Software modules generally can include instruction media storable within a memory location of an image processing apparatus and are typically composed of two parts. First, a software module may list the constants, data types, variable, routines and the like that can be accessed by other modules or routines. Second, a software module can be configured as an implementation, which can be private (i.e., accessible perhaps only to the module), and that contains the source code that actually implements the routines or subroutines upon which the module is based. The term “module” as utilized herein can therefore generally refer to software modules or implementations thereof. Such modules can be utilized separately or together to form a program product that can be implemented through signal-bearing media including transmission media and/or recordable media. An example of such a module that can embody features of the present invention is joint measurement software module 111 depicted in FIG. 1.

It is important to note that, although the embodiments are described in the context of a fully functional data-processing system (e.g., a computer system), those skilled in the art will appreciate that the mechanisms of the embodiments are capable of being distributed as a non-transitory and/or tangible program product in a variety of forms, and that the present invention applies equally regardless of the particular type of signal-bearing media utilized to actually carry out the distribution. Examples of signal bearing media include, but are not limited to, recordable-type media such as media storage or CD-ROMs and transmission-type media such as analogue or digital communications links.

FIG. 1 illustrates a data-processing apparatus 100, according to a preferred embodiment. Data processing apparatus 100 is utilized for accurately and reliably measuring joint angles. Data-processing apparatus 100 represents one of many possible data-processing and/or computing devices, which can be utilized in accordance with the disclosed embodiments. It can be appreciated that data-processing apparatus 100 and its components are presented for generally illustrative purposes only and do not constitute limiting features of the disclosed embodiments.

As depicted in FIG. 1, a memory 105, a processor (CPU) 110, a Read-Only memory (ROM) 115, and a Random-Access Memory (RAM) 120 are generally connected to a system bus 125 of data-processing apparatus 100. Memory 105 can be implemented as a ROM, RAM, a combination thereof, or simply a general memory unit. Joint measurement software module 111 includes software module in the form of routines and/or subroutines for carrying out features of the present invention and can be additionally stored within memory 105 and then retrieved and processed via a processor 110 to perform a particular task. A user input device 140, such as a keyboard, mouse, or another pointing device, can be connected to PCI (Peripheral Component Interconnect) bus 145. Joint measurement software module 111 is adapted to provide a graphical user interface 200 and processing the joint angle measurements. Processor 110 is adapted to process the joint angle measurements and body movement sensing.

Data-process apparatus 100 can thus include CPU 110, ROM 115, RAM 120, and a rendering device 190 (e.g., printer, copier, scanner, xerography equipment, etc.), which are also coupled to a PCI (Peripheral Component Interconnect) local bus 145 of data-processing apparatus 100 through PCI host-bridge 135. The PCI Host Bridge 135 can provide a low latency path through which processor 110 may directly access PCI devices mapped anywhere within bus memory and/or input/output (I/O) address spaces. PCI Host Bridge 135 also can provide a high bandwidth path for allowing PCI devices to directly access RAM 120.

A communications adapter 155, a small computer system interface (SCSI) 150, a raster image processor (RIP) 180, and an expansion bus-bridge 170 can also be attached to PCI local bus 145. The communications adapter 155 can be utilized for connecting data-processing apparatus 100 to a network 165, SCSI 150 can be utilized to control high-speed SCSI disk drive 160. An expansion bus-bridge 170, such as a PCI-to-ISA bus bridge, may be utilized for coupling ISA bus 175 to PCI local bus 145. Note that PCI local bus 145 can further be connected to a monitor 130, which functions as a display (e.g. a video monitor) for displaying data and information for a user and also for interactively displaying a graphical user interface (“GUI”) 200.

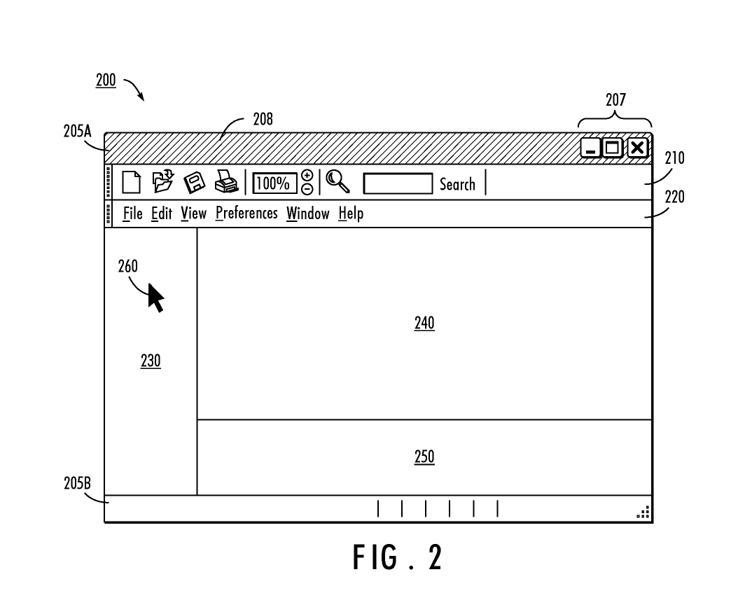

Referring to FIG. 2, illustrated is an exemplary GUI 200, according to an embodiment. GUI displays joint angle and body sensing measurements gathered by data processing system 100. Note that the term “GUI” generally refers to a type of environment that represents programs, files, options, and so forth by means of graphically displayed icons, menus, and dialog boxes on a computer monitor screen. A user actuates the appropriate keys on the user interface to adjust the parameters of the measurements. A user can access and operate the rendering device 190 using the GUI 200. The reasoning system can be a software module such as, for example, the joint measurement software module 111 of apparatus 100 depicted in FIG. 1.

The joint measurement software module 111, as disclosed herein, is configured to generate a GUI 200 on a display device. For example, the display device may include a cathode ray tube, liquid crystal display, plasma, or other display device. The GUI 200 may provide one or more windows or panes for displaying information to the user. The GUI 200 may be a window-like presentation defined by a top border 205A and bottom border 205B. Typical windows-like controls 207, include minimize, maximize, and close functions, may be provided at the upper-right hand corner (or at other locations) of the top border 205A. A menu bar 210 and tool bar 220 may be provided just below the top border 205A (or at other locations). The menu bar 220 may include a number of option menus, for example, File options, Edit options, View options, Preferences options, Window options, and Help options, etc. The tool bar 210 may include a number of features and options such as shortcut features to create a new file, open a file, save a file, print a file, a zoom feature, a magnification features, and a search feature. Many of features and options of the menu bar 220 and/or tool bar 210 may be conventional and/or customizable to support aspects of the application 100.

A user can interact with the GUI 200 to select and activate such options by pointing and clicking with a user input device such as, for example, a pointing device such as a mouse and/or with a keyboard. The GUI 200 controls the various display and input/output features of the application and allows a user to interact with the application 100 via a computer's operating system and/or one of more software applications. A pointer 260 may be provided to facilitate user interaction. For example, the user may use a mouse, joystick, light pen, roller-ball, keyboard, or other peripheral devices for manipulating the pointer 260 over the graphical user interface 200. Further, the pointer 260 may permit the user to navigate between the menu bar 220, the tool bar 210, and each of the panes 230, 240, 250 of the graphical user interface 200, as well as to select features and options from among various menus, “pop-up” windows, icons, prompts, etc. The graphical user interface 200 may include one or more active windows or panes.

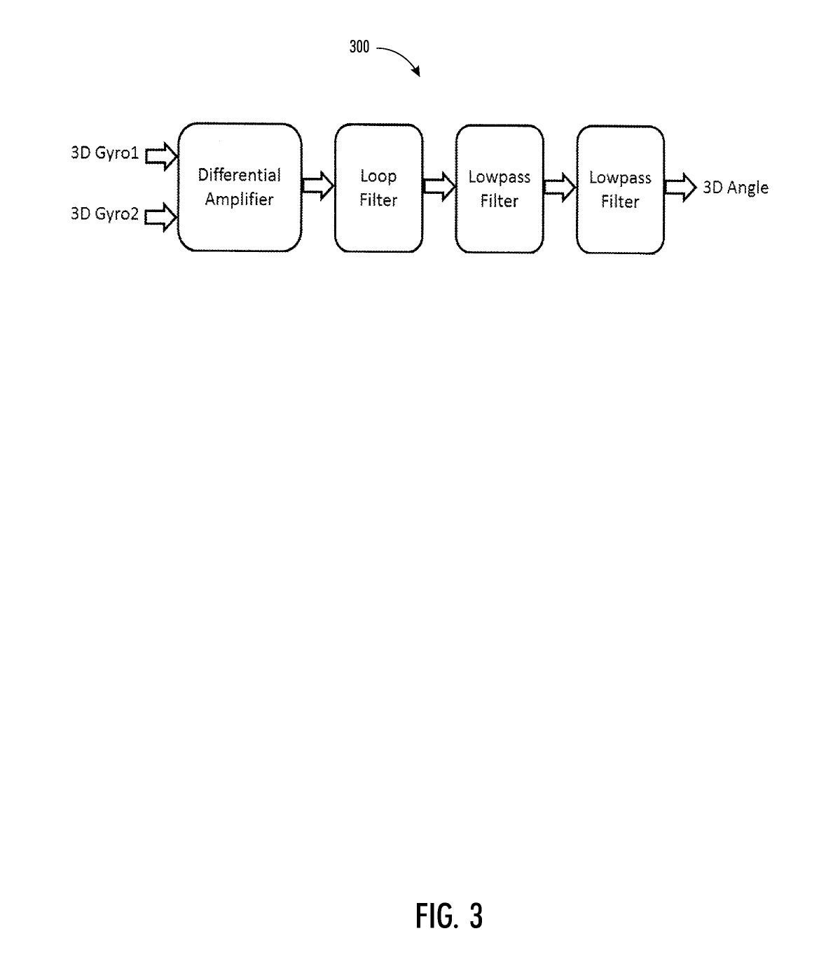

FIG. 3 illustrates an exemplary block diagram 300 displaying integration of the difference between two angular rate gyroscopes to provide three-dimensional joint angles in real-time, according to an embodiment. Three dimensional-gyroscope 1 and three-dimensional gyroscope 2 comprise a differential amplifier, loop filter, a first lowpass filter, and a second lowpass filter to accurately measure a three-dimensional joint angle. Modeling human spatially-oriented movement considers both the kinematics and kinetics of the various elements of the body and of the environment. The neuromuscular control system of the body integrates peripheral sensations relative to joint loads and processes these signals into coordinated motor responses. The position sense of a limb involves not only spindle receptors in the muscles, but also receptors in the skin and in the capsules of the joints. When tested with psychophysical means, the brain combines these many signals into an accurate position sense over a broad dynamic range. The control of a human's spatially-oriented behavior generally depends on internal reconstruction within the brain of gathered sensory information. Reconstruction can involve integration of physical movement including, for example, kinematics and kinetics, and interaction with the environment.

In an embodiment, the angular positions of the body's segments with respect to space and relative to each other, such as trunk vs. feet (“TF”) and head vs. trunk (“HT”), play an important role in understanding human dynamic behavior in space. This behavior is very complex and involves many physical, perceptual, and motor aspects.

Proprioception refers to the ability to know where a body part is located in space and to recognize movements of body parts (such as fingers and toes, feet and hands, legs and arms). One such body movement is a head rotation. Head rotations in space can result from a variety of scenarios (e.g., movements of either the neck, or the legs, or the support). Kinesthesia is a related term and refers to the sensation by which position, weight, muscle tension, and movement are perceived. Proprioception refers to the conscious and unconscious appreciation of joint position, while kinesthesia refers to the sensation of joint velocity and acceleration.

As such, the concomitantly-arising vestibular signal has to be perceptually interpreted with the help of the proprioceptive signals of relative motion between the various body segments. Used as a gravitoinertial measuring device, the vestibular system informs the brain about head rotation in space. Anatomically, the vestibular system consists of two parts: the macular organs and the semicircular canals. Their functions are those of a three-dimensional accelerometer and a three-dimensional pyrometer (i.e., angular speedometer), respectively. By combining the vestibular head-in-space signal with a proprioceptive trunk-to-head signal, a reliable trunk-in-space signal is obtained. Reliable sensory signals provide appropriate information concerning the rotations of head-in-space, trunk-to-head from the proprioceptive signal, and trunk-in-space.

In an embodiment, goniometers measure the relative angles of various anatomical points such as joints of the body. Goniometers are also known as angle measuring devices, a pivotable potentiometer, an arthrometer, fleximeter, or pronometer. The disclosed joint angle sense apparatus measures joint angles in three-dimensional space. In another embodiment, the disclosed goniometer is embodied as a wearable, wireless-based non-intrusive system, for example. Embodiments of the disclosed invention can include a non-invasive, wearable, portable device that gathers and analyzes real-time quantitative joint angle measurements. Previously-proposed devises are rigid, uncomfortable on the human skin. In an embodiment, the joint angle measurements gathered by the disclosed goniometer exhibit are free from drifts, offsets, hysteresis, temperature compensations, and other mechanical factors. The disclosed embodiments can comprise sensors that measure joint angles about one axis, or an axial sensor, or full rotation around a plurality of endpoints, for example. The disclosed joint measuring device can be positioned such that its neutral bend axis is aligned with the neutral bend axis of the flex hinge.

The system converts the small change in sensor resistance produced during bending into a usable signal. The analog circuitry in the system conditions the sensor signals, multiplexes between sensor channels, performs fixed-gain amplification of the sensor signal, applies channel-specific amplification offset and gain correction, and converts the resulting analog signal to a digital value.

The system measures changes in bend sensor resistance using a bridge circuit. Each sensor provides two piezoresistive elements for the bridge; the other half of the bridge consists of two high-precision reference resistors. The reference resistors are part of every sensor bridge as an analog multiplexer is used to cycle through all of the sensors in turn. The output of the Wheatstone bridge circuit is conditioned with a single-pole low-pass filter with a 3 dB frequency of 100 Hz. The choice of cutoff frequency balances between noise rejection and frequency response requirements.

The disclosed joint measurement or motion-sensing system provides an indication of a longitudinal axial bending, twisting, or elongation from movement of at least one body part to which the sensing element is coupled. A transducer converts the measurable parameter of the flowable substance into a transducer output signal. The transducer output signal is indicative of the longitudinal axial bending, twisting, or elongation of the sensing element. The joint angle measurement, body part position, or joint movement information can be derived from the transducer output signal. An optical position determining or tracking device automatically ascertains the angular relation of an object with respect to a reference direction or plane, and transforms the angular relation into analogous electrical signals. In an embodiment, the joint measurement device is augmented with human performance, measurements, evaluation, and/or assessment.

Based on the foregoing, it can be appreciated that a number of different embodiments, preferred and alternative are disclosed herein. For example, in one embodiment a joint sensing apparatus is disclosed. The apparatus can include a means for measuring joint angles in three dimensional space and a wearable means for non-intrusive, wireless data collection and analysis. In some embodiments, the data collection and analysis can include real-time quantitative joint angle measurement. In another embodiment, the apparatus can further include a means to prevent at least one of: drifts, offsets, hysteresis, temperature compensations, and other mechanical factors, In other embodiments, the apparatus can include a means for integrating a difference between at least one angular rate gyroscope to provide three dimensional joint angle measurements in real-time. In yet another embodiment, the apparatus can include a means for improving or augmenting human performance evaluation and assessment of three dimensional joint angle measurements.

In yet another embodiment, a joint sensing method is disclosed that comprises the steps of: measuring joint angles in three dimensional space and utilizing a wearable means for non-intrusive, wireless data collection and analysis. In some embodiments, the data collection and analysis comprises real-time quantitative joint angle measurement. In some embodiments, the method can include a step for preventing at least one of: drifts, offsets, hysteresis, temperature compensations, and other mechanical factors. In another embodiment, the method can include a step for integrating a difference between at least one angular rate gyroscope to provide three dimensional joint angle measurements in real-time. In yet another embodiment, the method can include a step for improving or augmenting human performance evaluation and assessment of three dimensional joint angle measurements.

In other embodiments, a joint sensing system is disclosed that comprises a processor; a data bus coupled to the processor; and a computer-usable storage medium storing computer code, the computer-usable storage medium being coupled to the data bus. The computer program code can comprise program instructions executable by the processor to: measure joint angles in three dimensional space; and utilize a wearable means for non-intrusive, wireless data collection and analysis. In another embodiment, the data collection and analysis can comprise rear-time quantitative joint angle measurement. In yet another embodiment, the system can include program instructions to prevent at least one of: drifts, offsets, hysteresis, temperature compensations, and other mechanical factors. In other embodiments, the system can include program instructions to integrate a difference between at least one angular rate gyroscope to provide three dimensional joint angle measurements in real-time. In another embodiment, the system can include program instructions to improve or augment human performance evaluation and assessment of three dimensional joint angle measurements.

In yet another embodiment, a computer-usable tangible storage device storing computer program code for joint sensing is disclosed. The computer program code can comprise program instructions executable by a processor, with the program instructions comprising: program instructions to measure joint angles in three dimensional space; and program instructions to utilize a wearable means for non-intrusive, wireless data collection and analysis. In another embodiment, the data collection and analysis can comprise real-time quantitative joint angle measurement. In yet another embodiment, the computer-usable tangible storage device can include program instructions to prevent at least one of: drifts, offsets, hysteresis, temperature compensations, and other mechanical factors. In other embodiment, the computer-usable tangible storage device can include program instructions to integrate a difference between at least one angular rate gyroscope to provide three dimensional joint angle measurements in real-time. In another embodiment, the computer-usable tangible storage device can include program instructions to improve or augment human performance evaluation and assessment of three dimensional joint angle measurements.

In yet another embodiment, a computer-implemented joint sensing method is disclosed that comprises the steps of: a computer measuring joint angles in three dimensional space and the computer utilizing a wearable means for non-intrusive, wireless data collection and analysis. In some embodiments, the data collection and analysis comprises real-time quantitative joint angle measurement. In some embodiments, the computer-implemented method can include a step for preventing at least one of: drifts, offsets, hysteresis, temperature compensations, and other mechanical factors. In another embodiment, the computer-implemented method can include a step for integrating a difference between at least one angular rate gyroscope to provide three dimensional joint angle measurements in real-time. In yet another embodiment, the computer-implemented method can include a step for improving or augmenting human performance evaluation and assessment of three dimensional joint angle measurements.

It will be appreciated that variations of the above-disclosed and other features and functions, or alternatives thereof, may be desirably combined into many other different systems or applications. Furthermore, various presently unforeseen or unanticipated alternatives, modifications, variations or improvements therein may be subsequently made by those skilled in the art which are also intended to be encompassed by the following claims.

What is claimed is:

1. A joint sensing and measurement apparatus, comprising:
means for measuring a joint angle in three-dimensional space; and
a wearable means for non-intrusive., wireless joint angle data collection and analysis comprising a real-time quantitative joint angle measurement in three-dimensions.

2. The apparatus of claim 1 further comprising means to prevent at least one of drifts, offsets, hysteresis, temperature compensations, and other mechanical factors in said real-time quantitative joint angle measurement.

3. The apparatus of claim 1 further comprising means for integrating a difference between at least one angular rate gyroscope for three-dimensional joint angle measurements in real-time.

4. The apparatus of claim 1 wherein said means for measuring a joint angle in three-dimensional space comprises at least one of a goniometer, a pivotable potentiometer, an arthrometer, a fleximeter, and a pronometer.

5. The apparatus of claim 1 wherein said means for measuring a joint angle in three-dimensional space comprises at least one of a sensor that measures said joint angle about one axis, an axial sensor, and a sensor that measures said joint angle in full rotation around a plurality of endpoints.

6. The apparatus of claim 1 wherein said means for measuring a joint angle in three-dimensional space comprises deriving a joint angle measurement, body part position, or joint movement from a transducer output signal produced by said joint sensing and measurement apparatus.

7. The apparatus of claim 6 wherein said transducer output signal is indicative of at least one of longitudinal axial bending of said joint, twisting of said joint, or elongation of a sensing element of said joint sensing and measurement apparatus.

8. A joint sensing system, comprising:
a processor;
a data bus coupled to said processor; and
a computer-usable non-transitory storage medium embodying computer code, said computer-usable non-transitory storage medium being coupled to said data bus, said computer program code comprising program instructions executable by said processor, said program instructions comprising:
program instructions to measure a joint angle in three-dimensional space; and
program instructions to non-intrusively collect and analyze wireless joint angle data collection and analysis comprising a real-time quantitative joint angle measurement in three-dimensions.

9. The system of claim 8 further comprising program instructions to prevent at least one of drifts, offsets, hysteresis, temperature compensations, and other mechanical factors in said real-time quantitative joint angle measurement.

10. The system of claim 8 further comprising program instructions to integrate a difference between at least one angular rate gyroscope for three-dimensional joint angle measurements in real-time.

11. The system of claim 8 further comprising at least one of a goniometer, a pivotable potentiometer, an arthrometer, a fleximeter, and a pronometer coupled to said processor to measure said joint angle in three-dimensions.

12. The system of claim 8 further comprising a sensor coupled with said processor, said sensor comprising program instructions to measure said joint angle about one axis and a sensor that measures said joint angle in full rotation around a plurality of endpoints.

13. The system of claim 8 further comprising program instructions to derive a joint angle measurement, body part position, or joint movement from a transducer output signal coupled to said processor.

14. The system of claim 13 wherein said transducer output signal is indicative of at least one of longitudinal axial bending of said joint, twisting of said joint, or elongation of a sensing element of said joint sensing and measurement apparatus.

15. A computer-usable non-transitory storage device storing computer program code for joint sensing, said computer program code comprising program instructions executable by a processor, said program instructions comprising:
program instructions to measure a joint angle in three-dimensional space; and
program instructions to non-intrusively collect and analyze wireless joint angle data collection and analysis comprising a real-time quantitative joint angle measurement in three-dimensions.

16. The computer-usable non-transitory storage device of claim 15 further comprising program instructions to prevent at least one of drifts, offsets, hysteresis, temperature compensations, and other mechanical factors in said rear-time quantitative joint angle measurement.

17. The computer-usable non-transitory storage device of claim 15 further comprising program instructions to prevent at least one of: drifts, offsets, hysteresis, temperature compensations, and other mechanical factors.

18. A computer-usable non-transitory storage device of claim 15 further comprising program instructions to integrate a difference between at least one angular rate gyroscope for three-dimensional joint angle measurements in real-time.

19. A computer-usable non-transitory storage device of claim 15 further comprising program instructions to measure said joint angle about one axis and a sensor that measures said joint angle in full rotation around a plurality of endpoints.

20. A computer-usable non-transitory storage device of claim 15 further comprising
program instructions to derive a joint angle measurement, body part position, or joint movement from a transducer output signal coupled to said processor, wherein said transducer output signal is indicative of at least one of longitudinal axial bending of said joint, twisting of said joint, or elongation of a sensing element of said joint sensing and measurement apparatus.
